(19) United States
(12) Patent Application Publication
(10) Pub. No.: US 2007/0088319 A1
(43) Pub. Date: Apr. 19, 2007

(54) Braided minimally invasive channel

(75) Inventor: Stephen Martone, Nashua, NH (US)

Correspondence Address: WOLF, BLOCK, SCHORR & SOLIS-COHEN LLP, 250 PARK AVENUE, NEW YORK, NY, 10177, US

(73) Assignee: Vison-Sciences, Inc., Natick, MA (US)

(21) Appl. No.: 10/572,188

(22) PCT Filed: Sep. 20, 2004
(86) PCT No.: PCT/US04/30807

§ 371 Date: Dec. 12, 2006

Related U.S. Application Data
(60) Provisional application No. 60/503,780, filed on Sep. 18, 2003.

(51) Int. Cl. A61M 31/00

(52) U.S. Cl. 604/508; 604/527; 606/108

(57) ABSTRACT

A probe assembly including an elongate tube for insertion into a body cavity, having a longitudinal central axis and a braided tube comprising a braid, coupled to the elongate tube, within the elongate tube or along side but not surrounding the elongate tube, wherein a central longitudinal axis of the elongate tube does not coincide with the central axis of the braided tube.

### RELATED APPLICATIONS

This application claims the benefit under 119(e) of U.S. provisional application 60/503,780, filed Sep. 18, 2003, the disclosure of which is incorporated herein by reference.

### FIELD OF THE INVENTION

The present invention relates to channels of minimally invasive medical tools, such as catheters and endoscopes.

### BACKGROUND OF THE INVENTION

In minimally invasive procedures, a catheter, endoscope or other elongate probe is inserted into a patient cavity, for diagnosis and/or treatment. In order to allow insertion of the elongate probe into the patient, the probe should be flexible, but strong, in order to resist stretching under tension and collapsing under compression. In addition, the elongate probe should be kink resistant. Also, in some cases it is desired that the elongate probe transfer a torque applied at its proximal end to the distal end.

U.S. Pat. No. 6,554,820 to Wendlandt et al., the disclosure of which is incorporated herein by reference, describes a composite medical tube that includes one or more coils and braids, which strengthen the tube. The braids and/or coils are embedded, at least partially, in an elastomeric material. A smooth inner layer formed of Teflon or a silicon based material is disposed on the radially inner surface of the channel so that medical tools inserted in the channel are surrounded by a smooth surface.

U.S. Pat. No. 5,483,951 to Frassica et al., the disclosure of which is incorporated herein by reference, describes working channels for a disposable sheath for an endoscope.

U.S. Pat. No. 6,440,124 to Esch et al., the disclosure of which is incorporated herein by reference, describes a delivery catheter formed of inner and outer tubes, which in one embodiment has an inner braided shaft.

Catheters having a braid embedded within a tube are described, for example, in U.S. Pat. No. 2,437,542 to Krippendorf, U.S. Pat. No. 6,143,013 to Samson et al., and U.S. Pat. No. 4,425,919 to Alston et al., the disclosures of which patents are incorporated herein by reference.

Some of these probes define a working channel through which medical tools and/or liquids may be delivered to a body cavity at the distal end of the elongate probe. The working channel is used, in some cases, to lead tools to a location in a patient to be treated. The inner surface of the working channel therefore is preferably slippery in order to allow smooth leading of tools to the distal end without getting stuck along the way. In order to be slippery, the material of the inner surface needs to be hard, a requirement that contradicts the need of the probe to be flexible. In some cases, therefore, a relatively rigid plastic extrusion tube, for defining a working channel, is inserted into the elongate probe after the elongate probe is located in the body cavity. This solution lengthens the minimally invasive procedure and is not always feasible.

In some cases, the working channel is used to apply suction to the treated location. For this functionality, the working channel should be strong enough so as not to collapse due to the suction.

Some elongate probes are not dedicated to a single task, but rather can be used for a plurality of different tasks. For example, an endoscope may have, in addition to a camera and wiring therefore, a working channel for introducing liquids to wash a window of the camera. The working channel is defined by an auxiliary tube, which may be located in the endoscope while the endoscope is inserted into the patient or may be inserted into the endoscope after the endoscope is within the patient.

In order to avoid contamination, disposable sheaths are used to cover elongate probes inserted into the patient. In some case, the sheaths include a tube that defines a working channel. Disposable sheaths are generally very floppy and require a working channel of complex construction in order to apply suction and/or deliver medical tools.

U.S. Pat. No. 6,293,909 to Chu et al., the disclosure of which is incorporated herein by reference, describes an endoscopic assembly covered by an expandable braided mesh sheath. The expendable mesh sheath may be expanded in order to anchor the assembly in a body cavity, take samples from the patient and/or dilate a stricture in the body cavity. When desired to replace an endoscope located within the mesh sheath while the sheath is in a patient, the endoscope is removed from the mesh sheath, the mesh sheath is expanded to make room for insertion of a different tool, such as a bougie and the other tool is then inserted to the mesh sheath.

### SUMMARY OF THE INVENTION

An aspect of some embodiments of the present invention relates to an auxiliary tube, for defining a working channel, formed at least partially by a braid or mesh. The auxiliary tube is used together with an elongate probe (e.g., an endoscope, ultrasound probe or catheter), such that during use, the auxiliary tube is within the elongate probe or along side the elongate probe. In some embodiments of the invention, the tube defining the working channel is located off-axis from the central axis of the assembly. Optionally, the tube is included within an outer sheath or other external tube that encompasses both the auxiliary tube and the elongate probe.

In some embodiments of the invention, the braid or mesh serves as a main structure element of the auxiliary tube, such that absent the braid the auxiliary tube would not exist. Optionally, the braid accounts for at least 80% or even 90% of the wall thickness of the auxiliary tube. In some embodiments of the invention, the braid allows at least some relative movement of its strands. Optionally, the auxiliary tube is flexible and does not include portions that break when they are collapsed or folded. Thus, the auxiliary tube may be folded into a closed state without breaking so that it can be reopened into an open state. In some embodiments of the invention, the braided tube is inserted into the patient in a folded state and is then opened for use, for example by passing a fluid or tool through the tube.

The auxiliary tube is either inserted into the patient together with the elongate probe or after the elongate probe is properly positioned, through a channel defined by the elongate probe.

In accordance with some embodiments of the invention, an auxiliary tube comprising a braid, is made sufficiently strong radially so that medical tools can be passed safely through the tube without risk of rupture of the tube, while still being flexible due to relative movement between the strands of the braid The flexibility is desired especially in those embodiments in which the auxiliary tube is inserted into the patient together with the elongate probe, so that the overall probe assembly inserted into the patient is easily maneuvered and causes less trauma to body cavities. In addition, even if the braided tube collapses it can still be used for passing fluids, e.g., for suction, due to the volume between the strands and/or the volume of an outer channel in which it is located, which is held open by the strands. Defining a working channel by a braided tube allows, in accordance with some embodiments of the invention, for easier collapsing of the tube when not in use, for limiting the cross-section of an assembly including an elongate probe and the working channel during insertion into the patient.

The braided tube is optionally sufficiently flexible so that it can enter body organs, such as the stomach, esophagus, bronchial passages, nasal cavity and/or urethra, through natural body cavities. In some embodiments of the invention, the braided tube is sufficiently flexible to undergo a bend of at least 60°, 90°, or even 180°, with a radius of less than 5 centimeters, 2.5 centimeters or even 1.5 centimeters, without a channel defined by the braided tube closing and/or kinking. The braid optionally comprises a relatively rigid material, such as polyester or nylon. In some embodiments of the invention, a solid tube, with similar dimensions, formed of the relatively rigid material is not flexible enough to bend to a same extent and with as small a radius, without a channel defined by the braided tube closing and/or kinking.

In some embodiments of the invention, the braided auxiliary tube is located within a channel defined by the elongate probe or a sheath covering the probe. In an exemplary embodiment of the invention, the elongate probe is surrounded by inner and outer sheaths and the braided tube is located between the sheaths. The braided tube is optionally a relatively durable structure positioned within an external channel defined between the sheaths. The braided tube thus prevents collapse of the channel, while it is used Alternatively, even when the braided tube collapses, the channel of the braided tube is not totally blocked, due to the braided structure. When the braid is not fluid-impervious, the advantages of the firm structure of the tube is enjoyed, while still enjoying the larger cross-sectional volume of the external channel, for example for suction.

An aspect of some embodiments of the invention relates to passing medical tools into a body cavity along walls of a working channel defined by a tube whose inner surface reflects the texture of a braid of the tube. Although a braid texture may be less desired than a smooth plastic extrusion, for inserting tools, the advantages of using a braid which is resilient and flexible while still being slippery outweigh the disadvantage of the braid texture.

Optionally, the inner surface of the tube is formed of an uncoated braid, so that the strands of the braid are allowed to flex freely relative to each other.

In some embodiments of the invention, a braid is inserted into a probe channel already located in the patient, to provide a lining required for better insertion of tools. Alternatively, a braided tube is inserted into a relatively large channel after the large channel is already located in a body cavity in order to better define the channel.

There is therefore provided in accordance with an embodiment of the invention, a probe assembly, comprising an elongate tube for insertion into a body cavity, having a longitudinal central axis and a braided tube comprising a braid, coupled to the elongate tube, within the elongate tube or along side but not surrounding the elongate tube, wherein a central longitudinal axis of the elongate tube does not coincide with the central axis of the braided tube. Optionally, the elongate tube comprises a catheter or an endoscope.

Optionally, the elongate tube comprises a sheath adapted for isolating an elongate probe from a patient's body. Optionally, the inner surface of the braided tube has a texture of the braid. Optionally, the inner surface of the braided tube comprises an uncoated braid surface. Optionally, the assembly includes a sheath adapted to receive the elongate tube and the braided tube adjacent each other.

Optionally, the assembly includes an internal sheath adapted to receive the elongate tube and an external sheath surrounding the internal sheath, wherein the braided tube is located between the internal and external sheaths. Optionally, the braided tube is more resilient than the internal and external sheaths. Optionally, the braided tube is permeable to liquids. Optionally, the axis of the braided tube is located outside the elongate tube.

Optionally, the braided tube has a substantially uniform cross-section substantially over its entire length. Optionally, the braided tube has different cross-section areas at different axial portions thereof. Optionally, the braided tube is flared at its proximal end. Optionally, the braided tube is stiffened by passing it through a heated die.

Optionally, at least a portion of the braided tube is coated by an adhesive or solvent.

Optionally, an end of the braided tube is coated by an adhesive or solvent, while a central portion of the braided tube is not coated. Optionally, the braided tube comprises strands which move independently relative to each other. Optionally, the braided tube is more flexible than the elongate tube. Optionally, the braided tube is sufficiently flexible to remain open even when bent at least 90° with a radius of less than 2.5 centimeters.

Optionally, the braided tube is formed of strands of a relatively rigid material, a solid tube of same dimensions as the braided tube formed of the relatively solid material would not remain open under a 90° bend with a radius of less than 2.5 centimeters.

Optionally, the braided tube is formed of strands of nylon or polyester. Optionally, the braided tube includes at least 36 strands. Optionally, the braided tube may be collapsed into a closed state and reopened into an opened state.

There is further provided in accordance with an embodiment of the invention, an invasive probe assembly, comprising an elongate tube for insertion into a body cavity; and a braided tube having an inner surface texture following a braid of the tube, the braided tube is coupled to the elongate tube, within the elongate tube or along side but not surrounding the elongate tube.

Optionally, the braided tube is substantially liquid-impervious. Optionally, the braid of the braided tube has holes between strands of the braid. Optionally, the braided tube is more flexible than the elongate tube. Optionally, the braided tube can apply suction along its length even in a collapsed state, due to spaces between strands forming the braid. Optionally, the braided tube is stiffened so as not to collapse under suction of at least 630 mm Hg. Optionally, the braided tube is stiffened by passing the tube through a heated die.

Optionally, the braided tube comprises a non-braided coating on its external and/or internal surface. Optionally, the braided tube does not have a non-braided coating on its internal surface.

There is further provided in accordance with an embodiment of the invention, a method of performing a medical procedure, comprising inserting a braided tube in a folded state into a cavity of a patient, opening the braided tube within the cavity and passing a tool or fluid through the opened braided tube. Optionally, opening the braided tube comprises passing a liquid or elongate tool through the braided tube.

There is further provided in accordance with an embodiment of the invention, a method of performing a medical procedure, comprising providing a braided tube having an inner surface texture following a braid of the tube, inserting the braided tube into a cavity of a patient; and passing a tool or fluid through the braided tube while it is in the cavity, such that the tool or fluid contacts the inner surface of the braided tube while being passed through the tube.

In some embodiments of the invention, passing the tool or fluid comprises passing a fluid. Optionally, passing the fluid comprises applying suction. Alternatively or additionally, passing the fluid comprises inserting a liquid through the braided tube into the body cavity.

There is further provided in accordance with an embodiment of the invention, a probe assembly, comprising an elongate tube for insertion into a body cavity and a braided tube comprising a braid, coupled to the elongate tube, within the elongate tube or along side but not surrounding the elongate tube, wherein the braided tube is more flexible than the elongate tube.

Optionally, the braided tube is sufficiently flexible to remain open even when bent at least 90° with a radius of less than 2.5 centimeters. Optionally, the braided tube is formed of strands of a relatively rigid material, a solid tube of same dimensions as the braided tube formed of the relatively solid material would not remain open under a 90° bend with a radius of less than 2.5 centimeters. Optionally, the braided tube is formed of strands of nylon or polyester.

There is further provided in accordance with an embodiment of the invention, a probe assembly, comprising an elongate tube for insertion into a body cavity; and a braided tube comprising a braid, coupled to the elongate tube, within the elongate tube or along side but not surrounding the elongate tube, wherein the braided tube comprises strands which move independently relative to each other.

Optionally, the braided tube comprises a braid of strands that is not coated over most of its length. Optionally, the braided tube comprises a braid which is coated at its end in order to prevent fraying.

There is further provided in accordance with an embodiment of the invention, a probe assembly, comprising an elongate tube for insertion into a body cavity and a collapsible braided tube comprising a braid, coupled to the elongate tube, within the elongate tube or along side but not surrounding the elongate tube, wherein the braided tube may be collapsed into a closed state and reopened into an opened state.

### BRIEF DESCRIPTION OF THE DRAWINGS

Exemplary non-limiting embodiments of the invention will be described with reference to the following description of the embodiments, in conjunction with the figures. Identical structures, elements or parts which appear in more than one figure are preferably labeled with a same or similar number in all the figures in which they appear, and in which:

FIG. 1A is a schematic side view of a sheath assembly including a braided tube defining a working channel, in accordance with an exemplary embodiment of the present invention;

FIG. 1B is a cross-sectional view of the sheath assembly of FIG. 1A, in accordance with an exemplary embodiment of the present invention;

FIG. 1C is a cross-sectional view of the sheath assembly of FIG. 1A, in a closed state, in accordance with an exemplary embodiment of the present invention;

FIG. 2A is a schematic illustration of a braided tube, in accordance with an exemplary embodiment of the invention;

FIG. 2B is a schematic sectional side view of a proximal end of an endoscope assembly, in accordance with an exemplary embodiment of the invention;

FIG. 3 is a schematic cross-sectional view of a sheath assembly, in accordance with another exemplary embodiment of the invention; and

FIG. 4 is a schematic cross-sectional view of a sheath assembly, in accordance with still another exemplary embodiment of the invention.

### DETAILED DESCRIPTION OF EXEMPLARY EMBODIMENTS

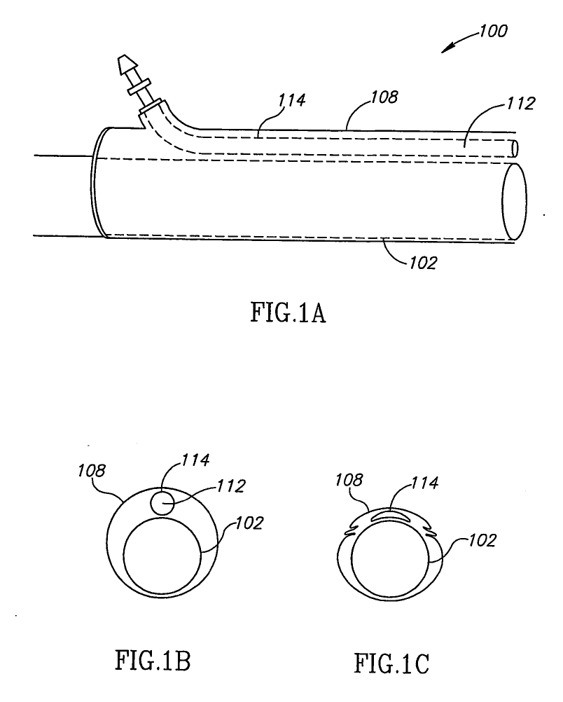

FIGS. 1A and 1B are a schematic side view and a cross sectional view of a sheath assembly 100, in accordance with an exemplary embodiment of the present invention. Assembly 100 optionally includes an annularly internal sheath 102 adapted to receive an endoscope and isolate the endoscope from the environment. An external sheath 108, having a larger circumference than internal sheath 102, optionally surrounds internal sheath 102. A braided tube 114, which defines a working channel 112, optionally runs along the outside of internal sheath 102, within external sheath 108. In some embodiments of the invention, channel 112 is used for introducing tools to the distal end of sheath assembly 100. Alternatively or additionally, channel 112 is used for applying suction and/or for introducing fluids to the distal end of sheath assembly 100. Channel 112 may have substantially any inner diameter suitable for its task, for example a diameter of about 2 mm, which is conventional in the art.

Braided tube 114 is optionally used to provide a working channel within the volume between sheaths 102 and 108. The use of a braided tube 114, rather than a plastic extrusion tube for example, provides for both flexibility and resilience due to the relative movement of strands of the braid, while still providing relative rigidity. The flexibility allows easier insertion of braided tube 114 into the patient, either together with assembly 100 or after the assembly is within the patient. Tube 114 optionally provides a slippery inner surface to facilitate easy insertion of tools. In addition, tube 114 has rigidity (e.g., radial strength) which provides some resistance to collapse of tube 114 which would block channel 112. In addition, even if tube 114 collapses, the volume between the strands of the tube would still allow passage of fluids along the tube and/or in a channel between sheaths 102 and 108 held open at least partially by tube 114. The structure of tube 114 is such that, in some embodiments of the invention, there is fluid connection between the inside of tube 114 and the channel between sheaths 102 and 108, outside tube 114.

FIG. 1C is a schematic cross-sectional illustration of sheath assembly 100 in a folded position, in accordance with an exemplary embodiment of the invention. It is noted that the elements in FIG. 1C are shown out of scale, for clarity. During insertion of an endoscope with sheath assembly 100 into a patient, braided tube 114 and external sheath 108 are optionally folded around internal sheath 102, such that the cross-sectional area of an endoscope with sheath assembly 100, during insertion, is not excessively enlarged by the inclusion of external sheath 108 and braided tube 114. Alternatively, braided tube 114 is kept open during insertion, unless sheath assembly 100 passes through a narrow body cavity where the tube is folded by contact with the walls of the narrow body cavity.

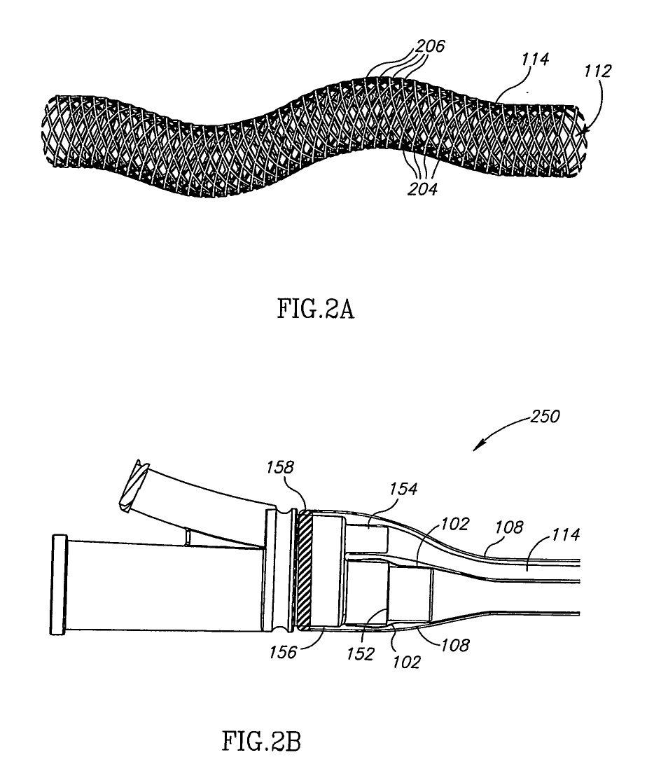

FIG. 2A is a schematic view of braided tube 114, in accordance with an exemplary embodiment of the present invention. Tube 114 comprises a plurality of strands 204 oriented in a first direction, woven into a plurality of strands 206 in an opposite direction, forming a mesh structure. In an exemplary embodiment of the invention, braided tube 114 includes between 24-48 groups of strands, for example 36 groups of strands. Alternatively, a larger number of groups of strands are used, for example between 48-64 groups of strands, or even more. In one particular embodiment of the invention, 52 groups of strands are used. The number of strands used is optionally selected according to the desired flexibility of tube 114 and the desired size of the inner lumen of the tube. Alternatively or additionally, any other braid structure known in the art may be used.

In some embodiments of the invention, the inner surface of braided tube 114 is rough, following the braiding structure of strands 204 and 206. When braided tube 114 is bent within a body cavity along with sheath assembly 100, the braided inner surface of tube 114 prevents medical tools and/or other accessory devices passing in channel 112 from getting stuck on the inner wall of the tube, since there is less contact area between the passing tools and the tube walls and hence there is less drag. Optionally, the material of the braid has a lower friction coefficient than sheath 108, to further facilitate the passage of tools through channel 112. To prevent snagging of the tools or accessories on the braid itself, the braid is optionally of a close weave, as indicated by the large number of strands.

In some embodiments of the invention, for simplicity of production, the inner surface of braided tube 114 is not coated, such that tools passing through the channel directly contact strands 204 and 206. Alternatively, the inner surface of braided tube 114 is coated with a very thin coating layer, which does not substantially affect the inner surface texture of braided tube 114. The coating may include, for example, polymeric coatings and/or oils which enhance lubricity. Alternatively, braided tube 114 is coated in order to limit the movement between the strands.

The strands are optionally free to move relative to each other, in order to achieve high flexibility of the tube. Alternatively, the strands are coupled to each other in order to limit the freedom of movement between the strands. Optionally, the inner surface of braided tube 114 is coated with an adhesive. Alternatively or additionally, the strands are at least partially fixed to one another by exposure to a solvent. In some embodiments of the invention, the coating is sufficiently flexible so that the entire tube flexes together. Alternatively, the coating is only loosely connected to the braid, so that the coating does not break when the braid is collapsed. Optionally, the coating of tube 114 forms a continuous surface. Alternatively, the coating covers only a portion of the surface area of the tube or the coating otherwise does not form a continuous surface.

In some embodiments of the invention, the inner surface of braided tube 114 is covered with a relatively thick coating (e.g., forming 10-20% of the thickness of tube 114) that provides a smooth inner surface of the walls. These embodiments may be used, for example, when required to enhance kink resistance at the expense of the advantages of having an inner braid texture with strands that move independently relative to each other.

In other embodiments of the invention, the strands are connected to each other at limited points along the length of tube 114, for example at crossing points of strands 204 and 206.

Alternatively or additionally to coating the inner surface of tube 114, the outer surface of tube 114 is coated. The coating optionally fills the interstices between the strands and prevents the strands from fraying.

In some embodiments of the invention, instead of coating the entire length of tube 114, the tube is coated at its distal and/or its proximal end, in order to prevent fraying of the braid. Particular coatings suitable for preventing fraying include adhesives, solvents and/or polymer solutions.

In an exemplary embodiment of the invention, an ultraviolet curable adhesive is used. Optionally, an adhesive compatible with the material of the strands of tube 114, e.g., forms a strong bond with the strands, is used. Alternatively or additionally, the adhesive used is selected to be compatible with the material of sheaths 102 and/or 108, e.g., the adhesive does not bond to the sheaths. Optionally, the adhesive used is selected according to the desired stiffness of tube 114. Optionally, a flexible adhesive, which is less traumatic to human anatomy, is used.

Tube 114 is optionally manufactured by creating a long braided tube and cutting the long tube into tubes 114 of predetermined lengths. Optionally, before performing the cutting, an area at which the cutting is to be performed is coated with an adhesive or solvent, to prevent fraying after the cutting. In some embodiments of the invention, the coating spans over less than 5 centimeters or even less than 2 centimeters around the area of the intended cutting. Alternatively to performing the coating before the cutting, the long tube is first cut into tubes 114 and then the ends of the tube are coated.

Alternatively or additionally to coating proximal and/or distal ends of braided tube 114, in some embodiments of the invention, tube 114 is coated in a predetermined middle section, where the tube is expected to require extra strength. For example, at points where tube 114 is planned to be exposed to particularly high abrasion and/or is planned to be used in a curved position.

The coating may make braided tube 114 liquid impervious, so that liquids passing within channel 112 do not penetrate the walls of the tube. It is noted, however, that as discussed below, in some embodiments of the invention, the walls of braided tube 114 are purposely pervious to liquids.

External sheath 108 optionally includes a relatively thin and flexible material so that it only minimally adds to the cross-sectional area of the sheath assembly when it is inserted into the patient In addition, the flexibility of external sheath 108 allows it to be expanded onto walls of a body cavity in which assembly 100 is located, in a manner which conforms to the shape of the body cavity.

Braided tube 114 optionally includes a more rigid material than external sheath 108 so as to define a channel of a required shape, according to the intended use of channel 112. The use of a relatively resilient tube to define a working channel allows relatively easy introduction of medical tools into the body cavity, without special means intended to prevent collapse of external sheath 108 onto the tool and/or twisting of the tool around internal sheath 102. In addition, absent braided tube 114, for example when external sheath 108 is partially connected to internal sheath 102 in order to define a channel, tools introduced in the volume between internal sheath 102 and external sheath 108 may get stuck in edges where the sheaths are connected or in contact.

Optionally, external sheath 108 and braided tube 114 have close or even same flexibility properties, although comprising different materials and having different structures. The braided structure of tube 114 optionally allows a similar flexibility to that of external sheath 108, although the strands of the braid comprise a more rigid material than sheath 108. In some embodiments of the invention, external sheath 108 comprises an elastic material, such as polyurethane or polyvinylchloride with a sufficiently large amount of added plasticizer, that can bend longitudinally around corners while the sheathed endoscope is inserted into the patient. Strands 204 and 206 comprise a more rigid, non-elastic plastic material, such as polyester, Polyethyleneterephtalate (PET), polyvinylchloride with a relatively small amount of added plasticizer, or a very thin (e.g., between about 0.05-0.1 mm) layer of Teflon or Polyethylene. In other embodiments of the invention, external sheath 108 comprises a more rigid material and accordingly strands 204 and 206 comprise materials more rigid than external sheath 108.

In an exemplary embodiment of the invention, strands 204 and 206 comprise polyester strands with a diameter of about 0.025 millimeters, such that braided tube 114 has a wall thickness of about 0.3-0.4 mm.

Alternatively to all of strands 204 and 206 having a same structure, different strands of braided tube 114 have a different thickness and/or material composition.

In some embodiments of the invention, strands 204 and 206 are tightly braided, without leaving substantial spaces between the strands, so as to substantially limit the fluid exchange between the interior and exterior of tube 114. Optionally, the spaces between the strands are smaller than the tool or tools to be passed through the tube 114. These embodiments are especially useful when tube 114 is primarily used for insertion of tools. In some embodiments of the invention, tube 114 is coated on its external surface with a fluid impervious coating, to prevent escape of liquids passed through channel 112. Such imperviousness for liquids is useful to prevent contamination of the endoscope in case inner sheath 102 is ruptured. Alternatively or additionally, the internal surface of braided tube 114 is coated with a fluid impervious coating.

In an alternative embodiment of the invention, strands 204 and 206 are loosely braided, leaving open spaces between adjacent strands. In accordance with this alternative, braided tube 114 may be used to keep external sheath 108 separated from internal sheath 102, so that suction can be applied to and/or liquids passed through the volume between the sheaths, without external sheath 108 collapsing onto internal sheath 102.

The extent to which braided tube 114 separates channel 112 from the area external to the tube may be the same over the entire length of the tube or may vary along the tube. For example, different coatings (including no coating at all) may be used for different longitudinal portions of the tube and/or the tightness of the braid may vary along the length of the tube.

In some embodiments of the invention, braided tube 114 is deformed in a non-self-collapsible manner, such that once opened, channel 112 does not close unless a force to induce the collapse is applied to the channel. For example, the material of braided tube 114 may be deformed in a predetermined shape, such that it does not collapse after being unfolded. Optionally, braided tube 114 is deformed over its entire length. Alternatively, braided tube 114 is deformed in one or more locations along its length, which locations are sufficient to prevent collapse of channel 112. In some embodiments of the invention, braided tube 114 is sufficiently strong so as not to collapse when suction of up to 620 mmHg, 635 mmHg or even 650 mmHg is applied through the tube. Alternatively, braided tube 114 is made sufficiently strong to withhold suction through the tube of pressures up to 700 mmHg or even up to 760 mmHg.

Further alternatively, stent-like structures are embedded within braided tube 114 along its length and/or are attached to its outer surface, in order to prevent collapse of tube 114 after it is unfolded. Further alternatively, braided tube 114 is formed in a self-collapsible manner, such that when not held open by external forces, channel 112 closes. For example, a braid in accordance with this alternative maybe produced by using a large number of strands, which are not heat set.

The deforming includes, in some embodiments of the invention, stiffening braided tube 114 using heat, to get an effect similar to ironing clothing. Optionally, braided tube 114 is passed through a heated die, having a cross-sectional shape at which tube 114 is to be stiffened. The heat of the die and/or the speed at which tube 114 is passed through the die are optionally adjusted according to the desired stiffness of tube 114. The temperature of the die is optionally selected so that the heat at most slightly deforms the strands but does not melt the strands causing them to bond together. In an exemplary embodiment of the invention, the die is heated to between about 275-310° F. (e.g., 290° F.), and tube 114 is passed through the die at a rate of between about 10-32 centimeters per second, e.g., 18 centimeters per second. Higher temperatures and/or slower passage rates are optionally used when a more stiff tube 114 is required. For example, a temperature at which crossing points of the strands melt into each other, may be used.

In some embodiments of the invention, the die has a circular cross-section, so that tube 114 maximizes its resistance to collapsing, for example when suction is applied through tube 114. Alternatively, the die has an oval cross-section, so as to reduce the cross sectional area of assembly 100, while maximizing the cross-sectional area of the tube. Further alternatively, the die has a triangular or rectangular cross-section, which allows stacking of a plurality of tubes next to each other while minimizing the space between them. In some of these embodiments of the invention, strands 204 and 206 comprise a thermoplastic material.

The passing of tube 114 through the die is optionally performed before coating the tube (or portions thereof), if the tube is coated, so that the heat does not adversely affect the coating. Alternatively, tube 114 is passed through the die only after the coating, when the heat may aid in melting the coating into the braided tube. In some embodiments of the invention, when tube 114 is produced by generating an elongate tube and cutting the tube, the tube is passed through the die before it is cut. Alternatively, tubes 114 are passed through the die after the elongate tube is cut.

FIG. 2B is a schematic sectional side view of a proximal end 250 of an endoscope assembly, in accordance with an exemplary embodiment of the invention. Proximal end 250 optionally includes a main channel port 152, an auxiliary channel port 154 and a general port 156. In mounting a sheath assembly (e.g., an assembly similar to assembly 100) onto proximal end 250, inner sheath 102 is mounted onto port 152, tube 114 is mounted onto port 154 and outer sheath 108 is mounted onto port 156. Optionally, a ring 158 holds outer sheath 108 in place. Alternatively or additionally, an adhesive is used to hold outer sheath 108 in place on its port. In some embodiments of the invention, sheaths 102 and 108 are made of thin and/or flexible materials, which are easily stretched to mount on their respective ports.

In some embodiments of the invention, the inner diameter of port 154 is at least the same size as the diameter of tube 114 along substantially its entire length, so that the entire capacity of tube 114 is usable through port 154. According to these embodiments, the outer diameter of port 154 is larger than the diameter of tube 114. In some embodiments of the invention, tube 114 is flexible enough to be stretchably mounted on port 154. Alternatively, tube 114 is flared at its proximal end, so that it fits on port 154. In some embodiments of the invention, tube 114 is flared at its proximal end in the braiding process, which has a varied diameter of braiding. Alternatively or additionally, the end of tube 114 is flared by stretching, for example by pushing the end of the tube over a mandrel. Optionally, the mandrel comprises a bullet shaped mandrel, which expands along its length. In some embodiments of the invention, the mandrel is heated. The mandrel is optionally heated to a temperature, which is suitable for both the flaring and for preventing fraying. Alternatively, the mandrel is kept at a low temperature not suitable for preventing fraying, for example in order to reduce production costs.

Alternatively or additionally, the distal end of tube 114 is flared. The flaring of the distal end of tube 114 is used, for example, when tube 114 is used for removing foreign objects from body cavities of the patient.

In other embodiments of the invention, the cross section area of tube 114 is substantially the same over its entire length. Port 154 optionally has, in accordance with this alternative, a smaller diameter than tube 114, to allow easy mounting of tube 114 on port 154. In other embodiments of the invention, instead of coupling tube 114 to port 154 by mounting an end of the tube onto the port, any other coupling method is used, such as inserting the end of tube 114 into port 154.

Further alternatively or additionally, tube 114 has a varying cross-section area over other portions of its length, for example at its distal end.

Alternatively to including a single braided tube 114 between inner sheath 102 and outer sheath 108, in some embodiments of the invention, a plurality of braided tubes are provided along sheath assembly 100. For example, one or more tubes may be used for tool insertion while another tube is used for suction. Further alternatively or additionally, one or more non-braided tubes (or a tube including an internal structural braid that does not affect the inner surface of the tube) are included in sheath assembly 100. In some embodiments of the invention, the volume between inner sheath 102 and external sheath 108 is used as an additional channel.

In the above description external sheath 108 and braided tube 114 are described as being in a collapsed state, while sheath assembly 100 is inserted into the patient. In other embodiments of the invention, only external sheath 108 is collapsed during the insertion into the patient, while braided tube 114 is open or partially open. Further alternatively, neither of external sheath 108 or braided tube 114 is collapsed while sheath assembly 100 is inserted into the patient.

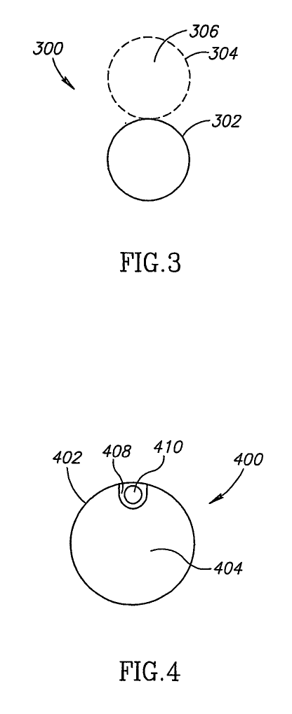

FIG. 3 is a schematic cross-sectional view of a sheath assembly 300, in accordance with an exemplary embodiment of the invention. In the embodiment of FIG. 3, a main sheath 302 is devised to receive an endoscope. An additional braided tube 304 defining a channel 306 is mounted on a side of main sheath 302. In the embodiment of FIG. 3, braided tube 304 is not surrounded by an external sheath.

In some embodiments of the invention, the width along which main sheath 302 and braided tube 304 are connected is relatively wide (e.g., close to the large end-to-end cross-sectional axis of channel 306). Alternatively, the width along which main sheath 302 and braided tube 304 are connected is relatively narrow, allowing both sheaths to define round channels. In some embodiments of the invention, channel 306 is designed to have a thin cross-section, for example with a generally elliptical shape, such that a relatively large channel cross sectional area can be achieved, without extending too far away from the body of the endoscope. Optionally, during insertion, braided tube 304 is folded or pleated, so as to minimize the cross-section of assembly 300 during insertion to the patient.

In this embodiment, the considerations on the extent to which braided tube 304 separates channel 306 from its exterior may be different from the considerations regarding braided tube 114, as braided tube 304 is not surrounded by an external sheath. When, for example, braided tube 304 is used for suction, it may be desired that braided tube 304 be fluid impervious, so that the suction reaches the distal end of channel 306.

FIG. 4 is a cross-sectional view of an endoscope sheath assembly 400, in accordance with an exemplary embodiment of the invention. Sheath assembly 400 comprises a sheath 402 shaped and sized to receive an endoscope (not shown) in a channel 404. In addition, sheath 402 defines a working channel 408. In order to allow for easier insertion of tools through working channel 408, a braided tube 410 is positioned within working channel 408. Braided tube 410 provides a resilient structure that reduces the chances that channel 408 will collapse. Optionally, braided tube 410 is not connected to working channel 408 but is rather kept in place due to its being within the working channel 408. Alternatively, braided tube 410 is connected to working channel 408 by an adhesive or any other suitable coupling means. In addition, a limited amount of suction can be applied through the spaces between the strands of braided tube 410 even if the braided tube collapses. Furthermore, as discussed above, braided tube 410 prevents tools passing through working channel 408 from puncturing sheath 402.

In some embodiments of the invention, braided tube 410 is located within working channel 408 when sheath assembly 400 is inserted into the patient. Alternatively, braided tube 410 is inserted into working channel 408 after assembly 400 is positioned within the patient. In some embodiments of the invention, in accordance with this alternative, any of the methods known in the art may be used to aid in leading braided tube 410 into working channel 408. In an exemplary embodiment of the invention, braided tube 410 is associated with a dove tail and sheath 402 defines a respective notch for receiving the dove tail, as described in PCT patent application PCT/US04/25238, filed Aug. 4, 2004, titled “Sheath with Channel for Endoscope”, and U.S. provisional patent application 60/491,971, filed Aug. 4, 2003, the disclosures of which are incorporated herein by reference. The dove tail may be braided along with tube 410 and/or may be connected to the braided tube with an adhesive or using any other attachment method.

Although working channel 408 is shown as completing a circular cross sectional shape of sheath assembly 400, sheath assembly 400 may have any other shape. For example, channel 404 for receiving the endoscope may have a circular shape, while working channel 408 bulges out of the circular shape.

Although the above description relates to a sheath assembly for an endoscope, the sheath assemblies of the present invention may be used with any other elongate probes, including ultrasound probes, catheters and other medical devices. Furthermore, the braided tube of the present invention may be used along with an elongate probe itself, rather than a sheath. For example, in the embodiments of FIG. 1A and FIG. 3, the internal sheath may be replaced by a catheter or other elongate probe, not necessarily covered by a sheath.

Furthermore, a braided working tube, with or without a leading element (e.g., a dove-tail), may be used together with substantially any sheath or non-sheathed medical probe.

It will be appreciated that the above-described methods may be varied in many ways, including changing sizes and materials used in forming the elements of the sheath assemblies. For example, the braided tubes of the present invention do not necessarily have a circular cross section, and may have other cross section shapes, such as elliptical. It should also be appreciated that the above described description of methods and apparatus are to be interpreted as including apparatus for carrying out the methods, and methods of using the apparatus.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art. Furthermore, the terms “comprise,” “include,” “have” and their conjugates, shall mean, when used in the claims, “including but not necessarily limited to.”

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

1. A probe assembly, comprising:
an elongate tube for insertion into a body cavity, having a longitudinal central axis; and
a braided tube comprising a braid, coupled to the elongate tube, within the elongate tube or along side but not surrounding the elongate tube, wherein a central longitudinal axis of the elongate tube does not coincide with the central axis of the braided tube.

2. An assembly according to claim 1, wherein the elongate tube comprises a catheter.

3. An assembly according to claim 1, wherein the elongate tube comprises an endoscope.

4. An assembly according to claim 1, wherein the elongate tube comprises a sheath adapted for isolating an elongate probe from a patient's body.

5. An assembly according to claim 1, wherein the inner surface of the braided tube has a texture of the braid.

6. An assembly according to claim 5, wherein the inner surface of the braided tube comprises an uncoated braid surface.

7. An assembly according to claim 1, comprising a sheath adapted to receive the elongate tube and the braided tube adjacent each other.

8. An assembly according to claim 1, comprising an internal sheath adapted to receive the elongate tube and an external sheath surrounding the internal sheath, wherein the braided tube is located between the internal and external sheaths.

9. An assembly according to claim 8, wherein the braided tube is more resilient than the internal and external sheaths.

10. An assembly according to claim 8, wherein the braided tube is permeable to liquids.

11. An assembly according to claim 1, wherein the axis of the braided tube is located outside the elongate tube.

12. An assembly according to claim 1, wherein the braided tube has a substantially uniform cross-section substantially over its entire length.

13. An assembly according to claim 1, wherein the braided tube has different cross-section areas at different axial portions thereof.

14. An assembly according to claim 13, wherein the braided tube is flared at its proximal end.

15. An assembly according to claim 1, wherein the braided tube is stiffened by passing it through a heated die.

16. An assembly according to claim 1, wherein at least a portion of the braided tube is coated by an adhesive or solvent.

17. An assembly according to claim 16, wherein an end of the braided tube is coated by an adhesive or solvent, while a central portion of the braided tube is not coated.

18. An assembly according to claim 1, wherein the braided tube comprises strands which move independently relative to each other.

19. An assembly according to claim 1, wherein the braided tube is more flexible than the elongate tube.

20. An assembly according to claim 1, wherein the braided tube is sufficiently flexible to remain open even when bent at least 90° with a radius of less than 2.5 centimeters.

21. An assembly according to claim 1, wherein the braided tube is formed of strands of a relatively rigid material, a solid tube of same dimensions as the braided tube formed of the relatively solid material would not remain open under a 90° bend with a radius of less than 2.5 centimeters.

22. An assembly according to claim 1, wherein the braided tube is formed of strands of nylon or polyester.

23. An assembly according to claim 1, wherein the braided tube includes at least 36 strands.

24. An assembly according to claim 1, wherein the braided tube may be collapsed into a closed state and reopened into an opened state.

25. An invasive probe assembly, comprising:
an elongate tube for insertion into a body cavity; and
a braided tube having an inner surface texture following a braid of the tube,
wherein the braided tube is coupled to the elongate tube, within the elongate tube or along side but not surrounding the elongate tube.

26. An assembly according to claim 25, wherein the braided tube is substantially liquid-impervious.

27. An assembly according to claim 25, wherein the braid of the braided tube has holes between strands of the braid.

28. An assembly according to claim 25, wherein the braided tube is more flexible than the elongate tube.

29. An assembly according to claim 25, wherein the braided tube can apply suction along its length even in a collapsed state, due to spaces between strands forming the braid.

30. An assembly according to claim 25, wherein the braided tube is stiffened so as not to collapse under suction of at least 630 mm Hg.

31. An assembly according to claim 25, wherein the braided tube is stiffened by passing the tube through a heated die.

32. An assembly according to claim 25, wherein the braided tube comprises a non-braided coating on its external surface.

33. An assembly according to claim 25, wherein the braided tube comprises a non-braided coating on its internal surface.

34. An assembly according to claim 25, wherein the braided tube does not have a non-braided coating on its internal surface.

35. A method of performing a medical procedure, comprising:
inserting a braided tube in a folded state into a cavity of a patient;
opening the braided tube within the cavity; and
passing a tool or fluid through the opened braided tube.

36. A method according to claim 35, wherein opening the braided tube comprises passing a liquid or elongate tool through the braided tube.

37. A method of performing a medical procedure, comprising:
providing a braided tube having an inner surface texture following a braid of the tube;
inserting the braided tube into a cavity of a patient; and
passing a tool or fluid through the braided tube while it is in the cavity, such that the tool or fluid contacts the inner surface of the braided tube while being passed through the tube.

38. A method according to claim 37, wherein passing the tool or fluid comprises passing a fluid.

39. A method according to claim 38, wherein passing the fluid comprises applying suction.

40. A method according to claim 38, wherein passing the fluid comprises inserting a liquid through the braided tube into the body cavity.

41. A probe assembly, comprising:
an elongate tube for insertion into a body cavity; and
a braided tube comprising a braid, coupled to the elongate tube, within the elongate tube or along side but not surrounding the elongate tube, wherein the braided tube is more flexible than the elongate tube.

42. An assembly according to claim 41, wherein the braided tube is sufficiently flexible to remain open even when bent at least 90° with a radius of less than 2.5 centimeters.

43. An assembly according to claim 41, wherein the braided tube is formed of strands of a relatively rigid material, a solid tube of same dimensions as the braided tube formed of the relatively solid material would not remain open under a 90° bend with a radius of less than 2.5 centimeters.

44. An assembly according to claim 41, wherein the braided tube is formed of strands of nylon or polyester.

45. A probe assembly, comprising:
an elongate tube for insertion into a body cavity; and
a braided tube comprising a braid, coupled to the elongate tube, within the elongate tube or along side but not surrounding the elongate tube, wherein the braided tube comprises strands which move independently relative to each other.

46. An assembly according to claim 45, wherein the braided tube comprises a braid of strands that is not coated over most of its length.

47. An assembly according to claim 45, wherein the braided tube comprises a braid which is coated at its end in a manner which prevents fraying.

48. A probe assembly, comprising:
an elongate tube for insertion into a body cavity; and
a collapsible braided tube comprising a braid, coupled to the elongate tube, within the elongate tube or along side but not surrounding the elongate tube, wherein the braided tube may be collapsed into a closed state and reopened into an opened state.
